(19) United States
(12) Patent Application Publication
(10) Pub. No.: US 2023/0181172 A1
(43) Pub. Date: Jun. 15, 2023

(54) HYDROGEL PRODUCING DEVICE FOR SAMPLING OF THE INTESTINAL LUMEN

(71) Applicant: Danmarks Tekniske Universitet, Kongens Lyngby (DK)

(72) Inventors: Sarvesh Kumar SRIVASTAVA, Kongens Lyngby (DK); Anja BOISEN, Kongens Lyngby (DK)

(73) Assignee: Danmarks Tekniske Universitet, Kongens Lyngby (DK)

(21) Appl. No.: 17/925,957

(22) PCT Filed: May 27, 2021
(86) PCT No.: PCT/EP2021/064225

§ 371 Date: Nov. 17, 2022

(30) Foreign Application Priority Data
May 27, 2020 (EP) 20176857.9

(51) Int. Cl. A61B 10/00

(52) U.S. Cl. CPC A61B 10/0096; A61B 10/0038

(57) ABSTRACT

The present disclosure relates to an ingestible device for sampling of the gastrointestinal tract. In particular, the device comprises an oral delivery capsule comprising a container having on the inside a hydrogel pre-polymer. The hydrogel prepolymer is capable of polymerising in situ, such as within the gastrointestinal tract, thereby collecting a sample of the gastrointestinal lumen by absorbing the lumen and its contents into the formed hydrogel.

### TECHNICAL FIELD

The present invention relates to an ingestible device for sampling of the gastrointestinal tract.

### BACKGROUND

Sampling of body lumens such as from the gastrointestinal tract have been performed using techniques such as sampling stool, mucosal brushing, and rectal swabs. Such technique, however, suffer from their inability to capture microorganisms throughout the intestinal tract, especially with respect to the ileum and jejunum. These techniques may also provide for poor spatial resolution of the sampling.

### SUMMARY OF THE INVENTION

The present inventors have conceived a device surprisingly efficient at collecting intestinal particles such as biomolecules and gut microbiome having a very narrow spatial resolution of the collection. The device is likewise able to collect sample from the ileum and/or the jejunum. Upon deployment of the device, it absorbs intestinal fluids and any particles part of the intestinal fluid in immediate vicinity of the device. Once deployment of the device has taken place, the absorbed particles remain immobile within the device until excretion.

The device comprises a hydrogel pre-polymer, which upon deployment of the device polymerises in situ and swells, thereby providing for absorption of intestinal particles such as biomolecules or gut microbiome into the device. The in-situ polymerisation of the hydrogel pre-polymer provides for a hydrogel with particularly desired properties, balancing sample amount uptake, stability of the hydrogel, and low exchange of the sample with the intestinal lumen. Most importantly, it allows enmeshing of intestinal particles such as biomolecules or gut microbiome inside the hydrogel matrix, thereby, preventing any cross-contamination.

One aspect of the present disclosure provides for an oral delivery capsule comprising:
a. a non-biodegradable, biocompatible open container with an extraction component and at least one opening;
b. a dry composition of a radical initiator and a hydrogel pre-polymer; and
c. an enteric coating;
wherein the dry composition is inside the container and the enteric coating covers the opening of the container.

One aspect of the present disclosure provides for an oral delivery capsule comprising:
a. a non-biodegradable, biocompatible open container with an extraction component and at least one opening;
b. a dry composition of a radical initiator and a hydrogel pre-polymer; and
c. an enteric coating and/or a lid;
wherein the dry composition is inside the container and the enteric coating covers the opening of the container.

In another aspect of the present disclosure, a non-biodegradable, biocompatible open container with at least one opening is provided, having a swollen hydrogel polymer comprising intestinal microbiome.

In another aspect of the present disclosure, a non-biodegradable, biocompatible open container with at least one opening is provided, having a swollen hydrogel polymer comprising intestinal microbiome and/or biomolecules.

In yet another aspect of the present disclosure, a use of an oral delivery capsule is provided, wherein the oral delivery capsule comprises a non-biodegradable, biocompatible open container with at least one opening, having on the inside a dry composition of a radical initiator and a pre-polymer, wherein the at least one opening of the open container and/or the capsule is covered in an enteric coating, for collection of the intestinal microbiome in a subject.

In a final aspect of the present disclosure, a method for collecting microbiome of the jejunum and/or ileum is provided, said method comprising the steps of:
a. providing an oral delivery capsule comprising a open container having on the inside a hydrogel pre-polymer and a radical initiator, wherein said container having on the inside a hydrogel pre-polymer and a radical initiator and/or the capsule is covered in an enteric coating;
b. ingesting the oral delivery capsule;
c. dissolving the enteric coating in the jejunum and/or ileum, effecting polymerisation of the hydrogel pre-polymer to produce a biocompatible hydrogel,
d. absorbing the microbiome present in the vicinity of the container into the hydrogel,
e. excreting the container comprising the hydrogel and absorbed microbiome, and
f. isolating the container comprising the hydrogel and absorbed microbiome from the excretion.

In a final aspect of the present disclosure, a method for collecting microbiome of the jejunum and/or ileum is provided, said method comprising the steps of:
a. providing an oral delivery capsule comprising a open container having on the inside a hydrogel pre-polymer and a radical initiator, wherein said container having on the inside a hydrogel pre-polymer and a radical initiator and/or the capsule is covered in an enteric coating and/or has a lid;
b. ingesting the oral delivery capsule;
c. dissolving the enteric coating in the jejunum and/or ileum, or opening the lid on the device effecting polymerisation of the hydrogel pre-polymer to produce a biocompatible hydrogel,
d. absorbing the microbiome present in the vicinity of the container into the hydrogel,
e. excreting the container comprising the hydrogel and absorbed microbiome, and
f. isolating the container comprising the hydrogel and absorbed microbiome from the excretion.

### DESCRIPTION OF DRAWINGS

FIG. 1: Oral administration in a capsule (101) via oral gavage. Capsule comprises container (102) having on the inside hydrogel pre-polymer (103) and radical initiator (104).

FIG. 2: Deployment of device in vivo produces hydrogel, which enmeshes intestinal content such as gut microbiota and biomarkers.

FIG. 3: One example of leading a container comprising the steps of adding PDMS (301), loading mixture (302), optional repetition of step 302 (303), loading of hydrogel pre-polymer (304), and stripping of PDMS mask (305).

FIG. 4: Deployment of device (402) triggered by exposure to aqueous environment (401) affects chemical activation by radical initiator (403) at 22 s followed by hydrogel formation (404) at 74 s.

FIG. 5: Resulting PEGDA hydrogel formation inside device leads to swollen hydrogel located both inside and outside of the container. Scale bar: 80 μm.

FIG. 6: ATR-FTIR spectra of real-time hydrogel formation. The not-yet-deployed device showed a peak at 2856 cm⁻¹ corresponding to unreacted (CH₂) terminal group of the PEGDA monomer. This peak disappears upon deployment of the device because the vinyl group is converted during polymerisation, initiated by addition of water. The intensity of stretching vibration around 1630 cm⁻¹ (v(C═C)) overlapped with OH angular deformation from water, which explains the existence of this band in the polymerized PEGDA spectrum (FIG. 6 inset). The band at 1635 cm⁻¹ corresponds to the reaction product of OH⁻ which comes from H₂O and oxidized products of ascorbate produced during the Fe³⁺ reduction. Polymer chain extension was further confirmed with the shifting of carbonyl band (around 1730 cm⁻¹) of the acrylate group (FIG. 6 inset). Finally, characteristic hydrogel swelling (OH peak) was confirmed at 3360 cm⁻¹ peak regain, growing gradually over a period of time (ca. 3 minutes). This peak was absent prior to the reaction.

FIG. 7: A: excised tissue of rat upper ileum region showing uniform distribution of administered devices. B: a deployed device shows interaction with the mucus layer. C: hydrogel formation inside tissue. Arrows mark distinct presence of PEGDA hydrogel.

FIG. 8: SEM images showing deployed devices from rat's ileum. Inset shows PEGDA hydrogel fibrils covering the device. Enmeshed gut bacteria are also visible as distinct rod-shaped bacteria trapped onto the device. Scale bar: 20 μm.

FIG. 9: Protein biomarker assessment. Amount of trypsin present in sample increases with increasing amount of devices, as assessed for two sets of retrieved devices (R1 and R2).

FIG. 10: Rarefaction curves for Sample 1 (gut tissue sample), Sample 2 (devices with scrapped mucus), and Sample 3 (devices washed).

FIG. 11: A half-capsule device that can seamlessly fit into a standard gelatin capsule-half. The standard gelatin capsule-half can be enteric-coated to disintegrate at specific pH of the GI tract, thereby, releasing the half-capsule device. A: schematic of a half—capsule design having pre-polymer loading chambers (1, 3) and a physical separator 2 having an opening. B & C: hydrogel formation of sodium polyacrylate and iron chloride/ascorbic acid in chambers 1 (B) and 3 (C), respectively. Light spots (indicated with white arrows) correspond to green fluorescence from fluorescent microparticles incorporated into the PBS buffer used for activation of hydrogel formation. The fluorescence demonstrates the particle trapping. An air bubble present in chamber 3 rose towards opening in separator 2 upon polymerisation, physically separating chambers 1 and 3, thereby minimizing cross contamination. Dashed line indicates position of opening.

FIG. 12: Dashed round shapes indicate openings. FIG. 12A shows a device featuring chambers 4 and 6 separated by separator 5. An internal funnel shaped feature (dashed, straight lines) facilitates loading of pre-polymer mixture inside the inner chamber 6. FIG. 12B shows a device featuring dissolvable blocks 8 preventing spring-based-bead closing mechanism 9 from closing against stoppers 7. Upon dissolution of blocks 8, spring-based-bead mechanism 9 closes against stoppers 7. The pre-polymer mixture can be loaded in the device above and/or below blocks 8. Upon entry of luminal fluids into device, pre-polymer mixture swells and/or polymerises to enmesh biomarkers and/or microbiome, as stopper 8 dissolves, to release the spring of spring-based-bead closing mechanism 9 and blocking the channel in stoppers 7. Polymerized and swollen hydrogel may create a positive pressure inside the device and may seal the opening to minimize cross-contamination during the transit of the GI tract. FIG. 12C shows a device with spring-based closing mechanism featuring lid 13 connected to stoppers 10 by extended springs 11, held extended by dissolvable blocks 12. Upon dissolution of blocks 12, lid 13 closes against stoppers 10. The tension and/or length of springs 11 can be varied to control the volume of the fluids entering in the inner chamber for polymerization. Hydrogel pre-polymer is present in the device under lid 13. Polymerized hydrogel creates a positive pressure inside the device and seals the entry points to minimize cross-contamination during the transit. FIG. 12D shows a device having one end-opening 14 and a solid base 17, a pre-polymerization mixture loading area 16, and a lid 15. Upon polymerisation and expansion of hydrogel pre-polymer, lid 15 closes against opening 14.

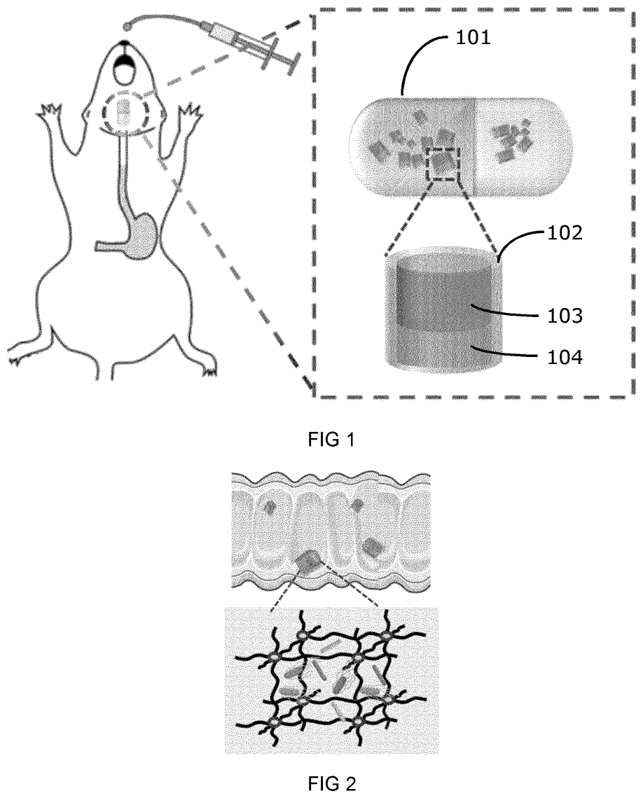

### DETAILED DESCRIPTION

Definitions

By “container” is meant an object which is capable of storing or holding matter or compositions of matter. As used herein, a container has at least one compartment which is suitable for storing or holding matter. The container does not necessarily comprise a lid. The container may however comprise a lid as disclosed herein. In one embodiment the lid may effect greater specificity and minimizing cross-contamination.

As used herein, the term “pre-polymer” means a compound or composition comprising a monomeric unit, an oligomeric unit, or a shorter polymeric unit, which may polymerise or further polymerise to form a polymer. Structurally, these moieties may comprise one or a plurality of chemical moieties which can polymerise. Such chemical moieties are for instance carbon-carbon double bonds. The terms “monomer”, “monomeric units” or “monomeric subunits” as used herein refer to all of the monomeric units, oligomeric units, or shorter polymeric units, which may polymerise or further polymerise to form a polymer.

As used herein, the term “dry” is taken to mean non-aqueous or substantially non-aqueous. “Substantially non-aqueous” refers to a content of water which is not sufficient to initiate deployment of the device, i.e. reaction between redox partners of the radical initiator, polymerisation of the hydrogel pre-polymer and/or swelling of hydrogel as outlined herein.

As used herein, the term “contrast agent” refers to a substance used to enhance or improve the visibility of internal bodily structures or objects in medical imaging. For example, the contrast agent is often used to enhance the visibility of the gastrointestinal tract. Examples of contrast agents are for instance: radiocontrast agents which refer to contrast agents used in X-ray imaging techniques, such as computed tomography (CT) and radiography (X-ray imaging); magnetic resonance imaging (MRI) agents which refer to a contrast agents used in MRI techniques; and ultrasonic imaging agents which refer to contrast agents used in sonographic diagnosis.

As used herein, the terms “absorb”, “adsorb”, “enmesh”, “encapsulate”, “trap” and “entrap” and other grammatical derived forms thereof, in connection with absorbing microbiome and other biomolecules into a hydrogel and/or polymer, are used synonymously herein.

By jejunum and/or ileum is also meant the upper and/or lower gastrointestinal tract. In one embodiment, the present disclosure provides for a device or capsule for sampling of the lower gastrointestinal tract. In one embodiment the present disclosure provides for a device or capsule for sampling of the upper gastrointestinal tract.

As used herein, the terms “hydrogel swelling”, “polymer swelling”, “polymerization”, “redox polymerization”, “free radical polymerization”, and “in situ polymerization” may be used synonymously to refer to the combination of one or more monomeric units (pre-polymers) and/or volumetric expansion of the materials disclosed herein.

As used herein, the terms “microbes”, “microbiome”, and “microbiota” are synonymous and comprises one or more species or strains of microscopic agents from the three domains of eubacteria, eukarya, and archaea as well as viruses, including culturable and non-culturable population.

As used herein, the terms “aqueous medium”, “gastric juices”, “luminal fluids”, “mucosal fluid” and terms derived therefrom are synonymous and comprises of liquids, digestive juices, enzyme cocktails, mucus, microbes, metabolites, cells, cells fragments, carbohydrates, fats, lipids, proteins, peptides, immune system molecules, immune system cells including chemokines and cytokines, blood, food particles or slurry thereof, acids, bases, dissolved gases, small molecules, hormones, nucleic acids, drugs, pro-drugs, drug metabolites and other molecules present in the GI tract from the mouth to the anus.

As used herein, the terms “sampling” and “collection” means obtaining part of the content of the GI tract, such as the aqueous medium, luminal fluid, and/or gastric juices as defined herein.

As used herein, the terms “analysis” and “analytical technique” refer to techniques comprising pH measurement, visual inspection, spectral analysis, pressure oxygen content, colorimetry, proteomic, metabolomics, mass spectroscopy (MS), nuclear magnetic resonance (NMR), chromatography, electrophoresis, assessing presence of haemoglobin, immunoassay, assessing protein-protein interactions, fluorescence, flow cytometry, assessing host microbiome interaction, assessing microbial metabolites, assessing host metabolites, nucleic acid hybridization, mRNA or cDNA transcription analysis, and sequencing of nucleic acids comprising of entire genomes, random fragments, or specific sections such as 16s rRNA of microbes and any combination of the techniques above for biological and chemical materials in the GI tract.

Device for Sampling of Gut Microbiome and Biomarkers in the Gastrointestinal Tract

The present disclosure provides for device for sampling of the gastrointestinal content. In particular, the device is useful for sampling of the intestinal content, such as the gut microbiome, biomolecules such as biomarkers forming part of the intestinal contents, and other particles forming part of the intestinal contents. The device is administered orally. Upon reaching the intestine, the device is deployed in response to external stimuli, whereby intestinal content in the immediate vicinity of the device is absorbed into parts of the device. Deployment of the device for example includes dissolution of enteric coating, initialisation of hydrogel pre-polymer polymerisation, or opening of lid.

The device absorbs intestinal contents substantially only in the time following immediately after deployment of the device, such as within seconds or minutes after deployment of the device. Subsequently, the absorbed sample of intestinal contents remain within the device, without any substantial exchange of the sample with the surrounding intestinal fluid. This allows for highly site-specific sampling of intestinal contents. Upon excretion of the device, the device is retrieved from the faeces. The retrieval is facilitated by an extraction component incorporated into the device. Accordingly, in one embodiment of the present disclosure, a device is provided capable of sampling gastrointestinal contents from the gastrointestinal tract. In a further embodiment of the disclosure, such gastrointestinal contents may comprise gut microbiome, biomolecules, or other particles forming part of the intestinal contents. In a specific embodiment, the device administered orally and deploys upon reaching a specific location in the gastrointestinal tract, after which the gastrointestinal content in the immediate vicinity of the device is absorbed into the device. In this specific embodiment of the disclosure, the device comprising the sample of gastrointestinal contents is retrieved from the faeces after excretion.

The device comprises a container having at least one opening. The opening may be controlled by a lid. The opening and the closing of said lid may be controlled by a lid closing and opening mechanism (lid closing mechanism or lid mechanism). The mechanism may be controlled by an actuator component. The actuator may comprise of a dehydrated sponge or superabsorbent material. Said mechanism may be activated or controlled via swelling of hydrogel and/or by a spring mechanism. The container is loaded with a dry composition comprising a radical initiator and a hydrogel pre-polymer. The device deploys when the radical initiator is exposed to water, such as water from an aqueous medium. Said aqueous medium is for example luminal or mucosal fluids. Specifically, upon exposure to water, the radical initiator dissolves and mixes with the hydrogel pre-polymer, which initiates polymerisation of the hydrogel pre-polymer. As the hydrogel pre-polymer polymerises, a hydrogel is formed. The hydrogel is hygroscopic and swells by absorbing aqueous medium in its vicinity, forming a swollen hydrogel. Any small particles which are part of the aqueous medium in the vicinity of the hydrogel are also absorbed into the hydrogel as it swells. Accordingly, one embodiment of the present disclosure provides for a device comprising a container having at least one opening, wherein the container is loaded with a dry composition comprising a radical initiator and a hydrogel pre-polymer.

The deployment of the device is triggered by an external stimulus. For instance, the device may be coated in an enteric coating to prevent aqueous medium from contacting the radical initiator. When the requirements for dissolution of said enteric coating are fulfilled, the enteric coating may dissolve, which lets the surrounding aqueous medium contact the radical initiator of the device. As the radical initiator dissolves, polymerisation of the hydrogel pre-polymer commences. Accordingly, in one embodiment of the present disclosure, the device comprises an enteric coating which separates the radical initiator from any aqueous medium surrounding the device. In a further embodiment, the enteric coating dissolves upon exposure to an external stimulus.

In the context of the disclosure, “deployment of the device” is taken to mean the sequence of events comprising dissolution of enteric coating, initiation of polymerisation of hydrogel pre-polymer, polymerisation of the hydrogel pre-polymer, swelling of hydrogel polymer, and absorption of aqueous medium in vicinity of the hydrogel.

The device herein comprises a biocompatible container. The hydrogel formed upon deployment of the device is likewise biocompatible.

Hydrogel Pre-Polymer and Hydrogel Polymer

In one embodiment of the present disclosure, the hydrogel pre-polymer comprises a single type of pre-polymer compound. The corresponding hydrogel polymer which forms upon deployment of the device is in this case a homopolymer. In another embodiment of the disclosure, the hydrogel pre-polymer is a composition comprising two or more pre-polymer compounds. The corresponding hydrogel polymer is in this case a copolymer.

The hydrogel pre-polymer compound comprises olefinic (i.e. unsaturated) monomers which are water-soluble. In one embodiment of the present disclosure, the monomer comprises a moiety selected from the group consisting of carboxylic acids, carboxylic acid anhydrides, sulfonic acids, dimethyl acrylamide, diacetone acrylamide, hydroxyl ethyl methacrylate, hydroxyl ethyl acrylate, dimethylaminoethyl acrylate, dimethylaminoethyl methacrylate, ethoxy ethoxy ethyl methacrylate, ethoxy ethyl methacrylate, methyl acrylate, ethyl acrylate, propyl acrylate, butyl acrylate, methyl methacrylate, and methacrylic acid. Further examples of such compounds are acrylic acid, methacrylic acid, maleic acid, cinnamic acid, itaconic acid, crotonic acid, ethacrylic acid, citoconic acid, mesaconic acid, fumaric acid, β-sterylacrylic acid, acrylate esters, acrylamides, olefins, vinyl esters, vinyl ethers, vinyl amides, 2-acrylamido-2-methylpropanesulfonic acid (AMPS), and 3-sulphopropyl acrylate (SPA). In a further embodiment of the present disclosure, the pre-polymer is a composition comprising two or more compounds having for example the above listed moieties. In a preferred embodiment of the disclosure, the pre-polymer comprise water soluble monomers having olefinic (i.e. unsaturated) moieties. Examples of such moieties are 2-acrylamido-2-methylpropanesulfonic acid (AMPS), acrylic acid, 3-sulphopropyl acrylate (SPA), and salts thereof. Particularly useful salts include, for example, the sodium, potassium, magnesium, calcium, lithium, and ammonium salts of the water-soluble monomers. In a preferred embodiment of the disclosure, the monomer comprises a moiety selected from the group consisting of acrylic acid, methacrylic acid, and 2-acrylamido-2-methyl propane sulfonic acid. In an even further preferred embodiment of the disclosure, the monomer is a diacrylate monomer. In a specific embodiment of the present disclosure, the monomer is poly(ethylene glycol) diacrylate (PEGDA). In one embodiment, the PEGDA has an average molecular weight of 300 to 850 g/mol, such as 400 to 750 g/mol, such as about 575 g/mol, such as 575 g/mol.

In one embodiment of the present disclosure, the hydrogel polymer comprises a crosslinked polymer. The cross-linked polymer may expand in volume upon exposure to fluids, such as aqueous medium.

The acidity of the medium in which polymerisation of the hydrogel pre-polymer and subsequent swelling of the formed hydrogel can affect the morphology of the swollen hydrogel. For some hydrogel pre-polymers, such as PEGDA, a light, voluminous morphology capable of absorbing a considerable about intestinal content is achieved by having a low pH value of the microenvironment in which polymerisation and swelling occurs. Furthermore, acidic pH facilitates radical generation, which in turn effects faster polymerisation. A low pH value can be achieved by loading an acid into the container. Accordingly, one embodiment of the present disclosure provides for a container having on the inside an acid. In a specific embodiment of the disclosure, the acid is an organic acid. In a further embodiment, the acid is ascorbic acid. The gut microenvironment may to a certain extent assist in achieving the acidic pH, as the gut microenvironment is slightly acidic, especially in the upper gastrointestinal tract (pH 5-6).

Using the in-situ polymerising hydrogel may be especially advantageous over employing a pre-formed hydrogel which merely swells in situ. The near-simultaneous or concurrent polymerisation and swelling of the hydrogel of the present disclosure may provide for absorption of a higher amount intestinal particles compared to a pre-formed hydrogel polymer. The in situ polymerisation and swelling may also provide for more inert hydrogels, i.e. hydrogels which have lower exchange with the surrounding intestinal fluids after deployment of the device. This in turn effects collection of intestinal particles such as microbiome in a narrow section of the intestinal with a minimum of contamination from the lumen downstream of the location of deployment of the device. This is due to the specific hydrogel morphologies obtainable through the combined polymerisation and swelling reactions that the device of the present disclosure provides for. Importantly, the in-situ polymerization facilitates highly localised enmeshing of gut microparticles within the hydrogel matrix and minimises cross-contamination when moving through the gastrointestinal (gi) tract.

Radical Initiator

The device of the present disclosure comprise a radical initiator, i.e. a free radical initiator. Such an initiator may be a water-soluble polymerisation initiator material. In one embodiment of the present disclosure, the radical initiator is a peroxygen compound such as a sodium, potassium or ammonium persulfate, caprylyl peroxide, benzoyl peroxide, hydrogen peroxide, cumene hydroperoxide, tertiary butyl diperphthalate, tertiary butyl perbenzoate, sodium peracetate, or sodium percarbonate. Metal ion oxidants are especially advantageous radical initiators because they offer better control of the radical generation. The metal ion oxidants may also be more biocompatible than other types of oxidants. In one embodiment of the present disclosure, the radical initiator is a metal ion oxidant such as Mn(III), Mn(VII), Fe(II), Fe(III) or Co(III). Redox systems comprising a plurality of components may also be utilised as radical initiators. In one embodiment of the present disclosure, the radical system comprises a peroxygen compound and a reducing agent. In a specific embodiment of the present disclosure, the radical system comprises a peroxygen compound and sodium bisulfite, L-ascorbic acid, or a ferrous salt. In a preferred embodiment of the disclosure, the radical initiator comprises iron(III)chloride and L-ascorbic acid.

Loading of the Device

The container of the present disclosure is loaded with a radical initiator and a pre-polymer. Herein below is described a selection of ways that the container may be loaded. However, it is to be understood in the context of the disclosure that different ways of loading the container will achieve similar outcome, namely formation of hydrogel polymer upon deployment of the device.

In one embodiment of the present disclosure, the radical initiator and the hydrogel pre-polymer are comprised within the container as a heterogeneous mixture.

As described herein, the container of the present disclosure may be shaped like a cylinder closed in one end such as to form a container with one opening. The container of the present disclosure may be loaded with first a layer of the radical initiator followed by a layer of the hydrogel pre-polymer. Thus, in one embodiment of the present disclosure a container is provided having on the inside a layer of radical initiator covered by a layer of hydrogel pre-polymer.

Container Material

The container of the present disclosure serves multiple purposes: i) it acts as a container to protect the loaded composition from the surrounding milieu, ii) it provides structural support to the device, iii) it acts as a receptacle for the extraction component, or alternatively acts as the extraction component, iv) it acts as a reaction chamber for the reactions between the radical initiator and the hydrogel pre-polymer, v) it guides swelling of the hydrogel, and vi) it retains the swollen hydrogel. It is therefore important that the container of the present disclosure remains intact from production of the device, through administration and deployment of the device, to retrieval of the device from excretion. Accordingly, in one embodiment of the present disclosure, the container is made from a non-biodegradable material.

While the container remains substantially inert from administration to excretion, it is paramount that the material from which the container is made does not elicit any toxic effect on the organism to which it is administered. Accordingly, the container of the present disclosure is made of a biocompatible material. For instance the material may comply with ISO 10993 and/or USP Class VI specifications for biocompatibility.

Different materials may be used individually or in combination for the container. In one embodiment of the disclosure, the material is a thermoplastic, fluoropolymer, elastomer, stainless steel, or glass. In another embodiment of the present disclosure the material may be a liquid silicone rubber material with a hardness level of 10 to 90 as determined using a durometer (e.g. MED-4942™ manufactured by NuSil™), a soft biocompatible polymer material such as polyvinyl chloride (PVC), polyethersulfone (PES), polyethylene (PE), polyurethane (PU) or polytetrafluoroethylene (PTFE), or a rigid polymer material coated with a biocompatible material that is soft or pliable, such as a poly(methyl methacrylate) (PMMA) or poly(lactic acid) (PLA) material coated with silicone polymer. In yet another embodiment, the material is a material commonly used in micro-fabrication, such as polydimethylsiloxane (PDMS), photoresists (like SU-8).

Use of different materials for different components may enable functionalisation of certain surfaces for interaction with microbes like bacteria and viruses, enzymes, antibodies, and other biomarkers. For example, the fluoropolymer Teflon (poly(tetrafluoroethylene)) may be used as a material in the ingestible device for movable components in order to reduce friction between these components.

For production purposes, it may be advantageous to produce the container of the present disclosure from a mouldable material. Accordingly, in one embodiment of the present disclosure, the container is made of a mouldable material such as a polymer. It may be advantageous that the container is made from a material which allows for easy provision of various container shapes and sizes, such as a material suitable for 3D printing, photolithography, or other means of microfabrication. Accordingly, in one embodiment of the present disclosure, the container is made of a material suitable for microfabrication.

Although specific materials are recited herein for construction of the container for illustrative purposes, the materials recited are not intended to be limiting, and one skilled in the art may easily adapt the device to use any number of different materials without affecting the overall operation or functionality of the device.

Container Structure

The container of the present disclosure comprises a compartment suitable for storing or holding matter or compositions of matter and at least one opening, wherein the at least one opening allows for access to the compartment. Described herein below are idealised container shapes. It is however to be understood that the shape of the container may differ from the described embodiments while maintaining its purposes as described herein.

In one embodiment of the present disclosure, the container is comprised of cylindrical walls, wherein one end of the cylinder is closed and one end of the cylinder is upon, i.e. a container having one opening. In another embodiment of the disclosure, the container is comprised of a cylinder having both ends open, i.e. a cylinder having two openings. Other structures may also be considered for the container, having one or more openings, such as boxes, spheres, or combinations of any of the mentioned structures, such as for example a cylinder capped with half-spheres, or a box having rounded edges and vertices.

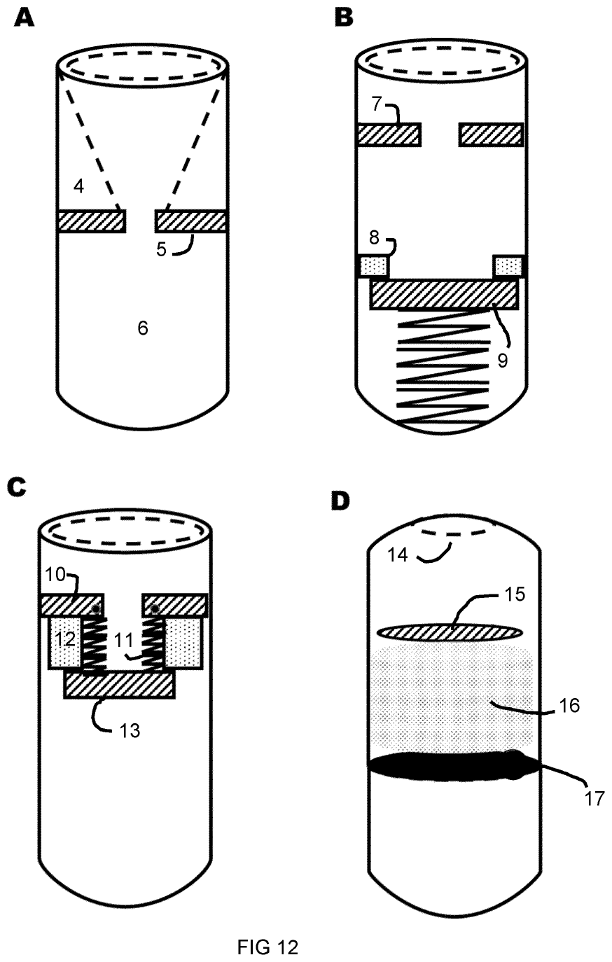

The delivery capsule of the present disclosure may further comprise a lid. Said the closing of said lid may be controlled by a lid closing mechanism. Such mechanism may be activated by one or more springs. Closing of said lid may be effected either by contraction or by expansion of said spring. The closing of said lid may be activated by dissolution of a dissolvable element, such as a dissolvable block. Exemplary constructs are shown in FIG. 12. Thus, one embodiment of the present disclosure provides for an oral delivery capsule having a lid. In a further embodiment, the lid is closed by a lid closing mechanism. In yet a further embodiment, the lid closing mechanism comprises a spring. In another embodiment, the lid closing mechanism comprises a dissolvable block. In one embodiment of the present disclosure, the dissolvable block comprises alum. In one embodiment of the present disclosure, the dissolvable block consists of alum.

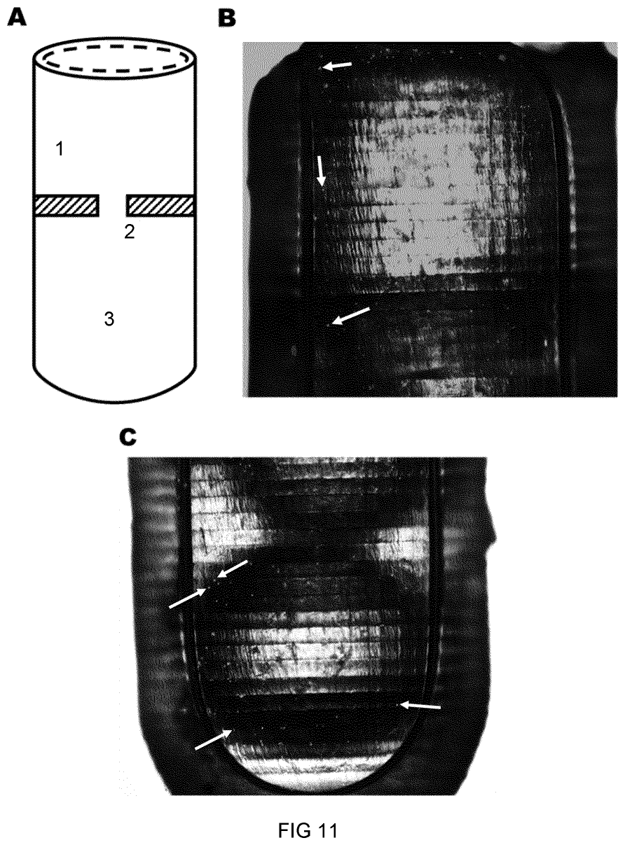

In a specific embodiment of the present disclosure, the oral delivery capsule comprises a separator, such as a bifurcation element. Exemplary separators are shown in FIGS. 11 and 12. In one embodiment, the separator has a hole or a channel. The polymerisation reaction described herein may produce a gas. Such gas may form one or more bubbles inside the container. One specific embodiment of the present disclosure provides for a cylindrical container having one closed end and one open end. The hydrogel pre-polymer may be loaded in the innermost chamber, i.e. the chamber on the opposite site of the separator relative to the opening. When such pre-polymer polymerises, gas may form which can form one or more air bubbles. The air bubble may be too large to pass through the hole or the channel in the separator, thus blocking the passage of liquid to and from the innermost chamber. This is further demonstrated in Example 8 herein. Further embodiments are described in Example 9 and shown in FIG. 12.

The oral delivery capsule of the present disclosure may comprise one or more containers loaded with a radical initiator and a hydrogel pre-polymer as described herein. However, in a preferred embodiment of the disclosure, the capsule comprise few containers, such as one container. It is preferred that capsules of the disclosure comprise few, large containers, rather than many small containers, as there are advantages associated with large containers. Such advantages include

Size of the Device

In one embodiment of the present disclosure, the oral delivery capsule has a length between 5 mm and 30 mm and a diameter between 1 mm and 13 mm. The container of the present disclosure may therefore be of similar dimensions as the capsule, such that the container may be comprised within the capsule. In one embodiment of the disclosure, the container is cylindrical and has a length between 5 and 30 mm and a diameter between 1 and 13 mm. In a specific embodiment of the disclosure, the capsule is for administration to a rodent, and the capsule has a length between 7 and 25 mm and a diameter between 1 and 3 mm. In a preferred embodiment of the disclosure, the capsule is for administration in a human subject, and the capsule has a length between 20 and 30 mm and a diameter between 9 and 13 mm, such as a length of 26 mm and a diameter of 11 mm.

The oral delivery capsule of the present disclosure may comprise one or more containers loaded with a radical initiator and a hydrogel pre-polymer as described herein. However, in a preferred embodiment of the disclosure, the capsule comprise few containers, such as one container. It is preferred that capsules of the disclosure comprise few, large containers, rather than many small containers, as there are advantages associated with large containers. Such advantages relates to the ease of which the device is retrieved from the faeces after excretion, namely that retrieving a single, larger device is more feasible than retrieving a plurality of smaller devices. The advantages also relate to the volume-to-surface area ratio of the formed, swollen hydrogel. In particular, a larger device will produce a swollen hydrogel with a higher volume-to-surface area than a small device. Having a high volume-to-surface area ratio is considered advantageous as this provides for less exchange of the adsorbed intestinal content after deployment of the device, compared to hydrogels having a low volume-to-surface area ratio. Additionally, using a single container within the capsule rather than multiple containers has the benefit that the sample collected is from the location in which the single device deployed. In contrast, if a capsule containing multiple loaded containers is administered and the multiple loaded containers deploys in different locations within the GI tract, the collected sample will be from the plurality of said locations in which the separate loaded containers deployed. This may be disadvantageous, if sampling from a single specific location within the GI tract is desired. Thus, in one embodiment of the present disclosure, the oral delivery capsule comprises a single container.

Alternatively, it may be desirable to sample from multiple locations within the GI tract. This can be achieved using a capsule having multiple loaded containers deploying in a plurality of locations within the GI tract. The deployment in different locations within the GI tract can be achieved by for instance coating the plurality of loaded containers with different enteric coatings which dissolve at different locations within the GI tract. The containers may comprise different extraction components or another type of visual identifier such as writing on the container, which correlate with the type of enteric coating employed, such that the containers upon extraction may be sorted depending on the enteric coating they originally were coated with. This has the benefit that a plurality of locations of the GI tract may be sampled using a single oral delivery capsule having on the inside a plurality of loaded containers. Accordingly, in one embodiment of the present disclosure, the oral delivery capsule comprises a plurality of containers.

Because the container is comprised within the oral delivery capsule, the size of the capsule dictates the upper limit for the size of the container. Accordingly, in one embodiment of the disclosure, the capsule is for administration to a rodent, and the container has a length between 7 and 25 mm and a diameter between 1 and 3 mm. In a preferred embodiment of the disclosure, the capsule is for administration in a human subject, and the container has a length between 20 and 30 mm and a diameter between 9 and 13 mm, such as a length of 26 mm and a diameter of 11 mm.

The container disclosed herein may be constructed as to slide inside one half of a standard size capsule such as a gelatine capsule. Capsules are typically constructed from a smaller-diameter “body” half that is typically filled and then sealed using a larger-diameter “cap”. Therefore, rather than encapsulating the entire container inside one capsule (body and cap), the present container may be constructed to have the shape and/or size of the “body” of a capsule, and then sealed with a “cap” of a capsule. Containers having this construction are referred to herein as “half-capsules” or “half-capsule containers”. Such containers may comprise one opening at one end, said opening sealed by a cap. The cap may comprise an enteric coating.

One embodiment of the present disclosure provides for an oral delivery capsule comprising a cylindrical container having an opening and having the diameter of the body of a standard capsule. In a further embodiment, the container comprises a capsule cap covering said opening. In yet a further embodiment, the cap comprises an enteric coating.

Contrast Agent

The oral delivery capsule of the present invention may comprise a contrast agent. An advantage of incorporating a contrast agent in the device is that the deployment of the device and/or the current location of the device in a subject can be monitored using medical imaging. For instance, incorporating the contrast agent into the container, such as encapsulating it fully or immobilising the contrast agent onto or within the container material allows for an observer to assess the current location of the device from administration to excretion. Accordingly, in one embodiment of the present disclosure, a container is provided having a contrast agent immobilised onto or into the container material. Alternatively, it may be advantageous to assess when and where in the intestinal tract that the device deploys. This can provide important information about which part of the intestinal tract is being sampled, which allows for better analysis, diagnosis, and treatment relating to the sampled intestinal contents. Visualisation of the location of deployment may be achieved by incorporating the contrast agent in a component of the device which undergoes chemical or mechanical change during deployment of the device. Accordingly, the contrast agent may for instance be incorporated into or adsorbed onto the capsule, the radical initiator or the hydrogel pre-polymer. Alternative, the contrast agent may be loaded into the container such that it is released from the container as the device deploys. Thus, in one embodiment of the disclosure, the device comprises a contrast agent which is released, partly or fully, from the device upon deployment of the device. Examples of contrast agents which are considered useful in the present disclosure are barium based contrast agents such as barium sulfate, iron-based compounds, and gadolinium based contrast agents such as gadopentetic acid.

Extraction Component

The device of the present disclosure comprise an extraction component. The extraction component facilitates retrieval from the faeces once the device is excreted. The extraction component may be a discrete component incorporated into the container material of the device. Alternatively, the extraction component may be an integrated feature of the container, such that the entire container constitutes the extraction component.

In one embodiment of the present disclosure, the extraction component comprises a magnetic material, such as a magnetic metal. Magnetic materials are attracted to external magnetic fields, such as that exerted by a magnet. Accordingly, an extraction component comprising a magnetic material, such as a magnetic metal, incorporated into the container of the device, may facilitate extraction of the container from excreted faeces using an external magnet. Specifically, the external magnet may be positioned close the faeces comprising the excreted container having the extraction component. On account of the magnetic material incorporated in the container being attracted to the magnet, the container can be retrieved from the faeces.

In one embodiment of the present disclosure, the extraction component comprises a container having a colour which is easily distinguishable from the colour of faeces. This facilitates visual recognition of the container with the excreted matter. Colours which are deemed easily distinguishable from the colour of faeces will typically have one or more of the following features: colours having a high value (also referred to as pure colours, or colours having no tint), optionally also having high saturation; bright colours; and colours colloquially referred to as “neon colours”. Examples of colours deemed useful as extraction components are: bright green, cyan, pink, and yellow. In one embodiment of the present disclosure, the extraction component comprises a container having on the outside a single, solid colour. In another embodiment of the disclosure, the extraction component comprises a container having on the outside a combination of two or more colours. In the context of the extraction component comprising a container having a colour which is easily distinguishable from the colour of faeces, it is considered an advantage that substantially all of the outer surface of the container is coloured, as to facilitate visual recognition of the container within excreted matter.

Enteric Coating

The device of the present disclosure comprises an enteric coating. The role of the enteric coating is to separate the radical initiator from the aqueous milieu of the gastrointestinal tract, until deployment of the device is desired. Accordingly, in one embodiment of the disclosure, the enteric coating covers the capsule or the container having on the inside the radical initiator. Preferably, the enteric coating separates both the radical initiator and the hydrogel pre-polymer from the aqueous milieu of the gastrointestinal tract until deployment of the device is desired. Thus, in a preferred embodiment of the present disclosure, the enteric coating covers the capsule or the container having on the inside the radical initiator and the hydrogel pre-polymer.

The separation of the radical initiator from the aqueous milieu of the gastrointestinal tract by the enteric coating can be achieved by coating specific components of the device. In one embodiment of the disclosure, the container having on the inside the radical initiator and the hydrogel pre-polymer has its at least one opening covered by a layer of the enteric coating. In a further embodiment, the entire open container having on the inside a radical initiator and a hydrogel pre-polymer is coated in the enteric coating. In a different embodiment, the capsule comprising the open container is covered in an enteric coating. In yet a further embodiment, the device comprises different types of enteric coatings, such as layered enteric coatings or enteric coatings covering different components of the device. The use of different coatings may allow for deployment of the device in locations of the gastrointestinal tract otherwise not accessible with the use of a single enteric coating.

The enteric coating or enteric coatings of the present disclosure may dissolve in response to a change in pH of the surrounding aqueous milieu. In one embodiment of the present disclosure, the enteric coating may remain intact at pH values below 5, but dissolve at pH values above 6. Alternatively, the enteric coating may me stable for a certain amount of time, such as for a few or several hours, after which it dissolves.

It is to be understood that the enteric coating may separate the radical initiator from the aqueous milieu of the gastrointestinal tract in several different ways as outlined herein. These different ways of separating radical initiator and aqueous milieu of the GI tract using an enteric coating will collectively be referred to as “covering the opening of the container”.

Sampling of Gut Microbiome and Biomolecules.

The oral delivery capsule of the present disclosure comprise a hydrogel pre-polymer which upon deployment of the device polymerises and swells by absorption of the aqueous, intestinal content. The intestinal content contains particles such as gut microbiome and biomolecules. Any particles small enough to enter the hydrogel as it swells will be absorbed into the swollen hydrogel. Accordingly, one embodiment of the present disclosure provides for a non-biodegradable, biocompatible open container with at least one opening, having a swollen hydrogel polymer comprising intestinal particles. A further embodiment of the disclosure provides for a non-biodegradable, biocompatible open container with at least one opening, having a swollen hydrogel polymer comprising intestinal biomolecules such as biomarkers. A preferred embodiment of the disclosure provides for a non-biodegradable, biocompatible open container with at least one opening, having a swollen hydrogel polymer comprising intestinal microbiome.

The oral delivery capsule of the present disclosure is especially useful for sampling of the intestinal content, such as sampling of the intestinal content in a subject. Accordingly, in one embodiment of the present disclosure a use of an oral delivery capsule is provided for the collection of the intestinal content in a subject, wherein the oral delivery capsule comprises a non-biodegradable, biocompatible open container with at least one opening, having on the inside a dry composition of a radical initiator and a pre-polymer, wherein the at least one opening of the open container and/or the capsule is covered in an enteric coating. In a specific embodiment of the disclosure, the intestinal content is gut microbiome.

The present disclosure also provides for a method of collecting intestinal content, such as content in the jejunum and/or ileum. The intestinal content may be gut microbiome. Accordingly, in one embodiment of the present disclosure, a method is provided for the collection of intestinal content of the jejunum and/or ileum, said method comprising the steps of a) providing an oral delivery capsule comprising a open container having on the inside a hydrogel pre-polymer and a radical initiator, wherein said container having on the inside a hydrogel pre-polymer and a radical initiator and/or said capsule is covered in an enteric coating; b) ingesting the oral delivery capsule; c) dissolving the enteric coating in the jejunum and/or ileum, effecting polymerisation of the hydrogel pre-polymer to produce a biocompatible hydrogel; d) absorbing the intestinal content present in the vicinity of the container into the hydrogel; e) excreting the container comprising the hydrogel and absorbed intestinal content; and f) isolating the container comprising the hydrogel and absorbed intestinal content from the excretion. A preferred embodiment of the present disclosure provides for a method for collection of intestinal microbiome of the jejunum and/or ileum, said method comprising the steps of a) providing an oral delivery capsule comprising a open container having on the inside a hydrogel pre-polymer and a radical initiator, wherein said container having on the inside a hydrogel pre-polymer and a radical initiator and/or said capsule is covered in an enteric coating; b) ingesting the oral delivery capsule; c) dissolving the enteric coating in the jejunum and/or ileum, effecting polymerisation of the hydrogel pre-polymer to produce a biocompatible hydrogel; d) absorbing the intestinal microbiome present in the vicinity of the container into the hydrogel; e) excreting the container comprising the hydrogel and absorbed intestinal microbiome; and f) isolating the container comprising the hydrogel and absorbed intestinal microbiome from the excretion.

The oral delivery capsule of the present disclosure may be useful for sampling of the colon. Accordingly, one embodiment of the present disclosure provides for an oral delivery capsule for sampling of microbiome of the colon.

One embodiment of the present disclosure provides for a method for sampling microbiome and biomolecules in vivo, said method comprising
a. polymerising a hydrogel pre-polymer in the gut, thereby absorbing microbiome into the polymerised hydrogel, and
b. collecting the polymerised hydrogel.

One embodiment of the present disclosure provides for a device comprising:
a. a non-biodegradable, biocompatible open container with an extraction component and at least one opening;
b. a dry composition of a radical initiator and a hydrogel pre-polymer; and
c. an enteric coating.

One further embodiment provides a device having a lid opening and closing mechanism. One further embodiment provides a device having an actuator component controlling the lid opening and closing mechanism. In one embodiment, the actuator component comprises a dehydrated sponge. In one embodiment, the actuator component comprises a super absorbent material. In one embodiment, the actuator component comprises a spring.

### EXAMPLES

### Example 1: Fabrication of Devices

The epoxy based negative photoresist SU-8 (MicroChem, USA) was used to fabricate the containers (hollow cylindrical structures) using a two-step photolithography technique. The first layer of SU-8 (2035, 69.95 wt %) was spin coated (Suss MicroTec RCD8 with Gyrset) on a fluorocarbon coated silicon wafer and soft baked at 50° C. for 2 h (temperature ramping at a rate of 2° C./min) and slowly cooled down to room temperature. The bottom of the containers (thickness 35 μm) was defined using UV exposure for 30 sec using soft contact (Suss Mask Aligner MA6, equipped with in-line notch filter) as well as two bursts of 250 mJ/cm². A post exposure baking was conducted at 50° C. for 6 h (with the same ramping conditions as mentioned before). The second layer of resist (SU-8 2075, 73.45 wt %) was spin coated and prebaked at 50° C. for 10 h to define the well wall (height 220 pm) using the same temperature ramping condition. A global WEC chuck was used to conduct the UV exposure in proximity mode, followed by two bursts of 250 mJ/cm² for 30 sec, after which, a post exposure baking was performed at 50° C. for another 10 h using the same temperature ramping condition. The resulting substrate was developed in mr-Dev 600 for 2×20 minutes. Then the resist on the substrate was flushed with isopropanol and left to dry before inspection. The silicon wafer was cleaved into several rectangular slides, each containing 625 containers. The inner height and inner diameter of the resulting containers were 220 μm and 190 μm respectively, with a 25 μm thick sealed bottom. Diced chip was teared off and placed in a sample holder. A very thin layer of PDMS mixture acting as a negative mask (PDMS monomers mixed with Iguracure D2959 at the volume ratio of 10:1) was spread across the spaces between the wells,. Then PDMS was fully cured in the oven at 37° C. overnight.

### Example 2: Loading of Devices

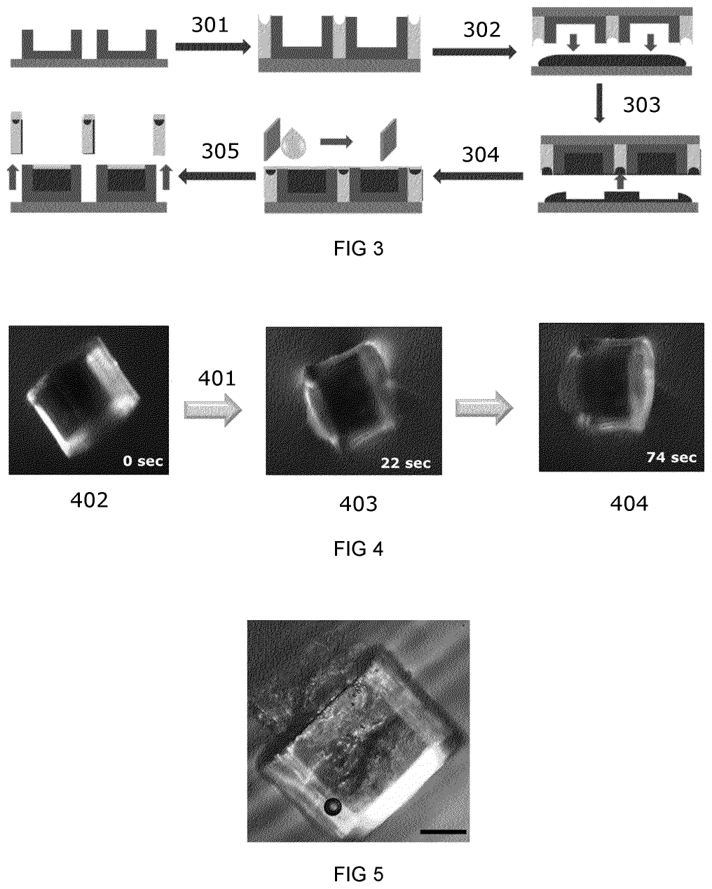

Ascorbic acid and FeCl₃ (iron (III) chloride, iron trichloride) serving as redox radical initiators, were mixed uniformly in a weight ratio of 5:1 (wt./wt.) via a mortar and pestle and stored in a vacuum chamber at room temperature. A modified brush loading method was used to load the resulting reactant mixture into the devices (with PDMS negative mask). Then a thin layer of PEGDA monomer (around 20 μL, mw 575 g/mol) was evenly spread over the containers using doctor blading technique. The resulting devices was dried and kept in a dry environment. Lastly, the negative PDMS mask was peeled off followed by the characterization and in vitro and in vivo studies. The procedure for loading the devices is outlined in FIG. 3.

Rhodamine 6G dye (R6G) (Sigma-Aldrich, Danmark) can optionally be used without any pre-treatment to stain PEGDA hydrogel for easy visualization of the devices. R6G was distributed in radical initiators at a weight ratio of 1:50 (wt./wt.) using a mortar and pestle. The resulting mixtures were loaded in to the devices, covered with a layer of PEGDA monomer via the same methods described above. The loaded devices were dried at 40° C. for 24 hours on a hot plate. A Carl Zeiss optical microscope Scope A1 coupled with brightfield, darkfield and C-DIC mode was used to visualize device activation.

Fluorescein isothiocyanate (FITC)-insulin was mixed with radical initiator agents at a weight ratio of 1:50 and loaded into containers to fabricate the devices using the same method as was used to prepare R6G containing devices. The chip with FITC-insulin devices was put in a plastic dish and wrapped with tinfoil. 20 min UV irradiation was used to sterilize the devices prior to cell studies.

### Example 3: PEGDA Hydrogel Formation and Swelling

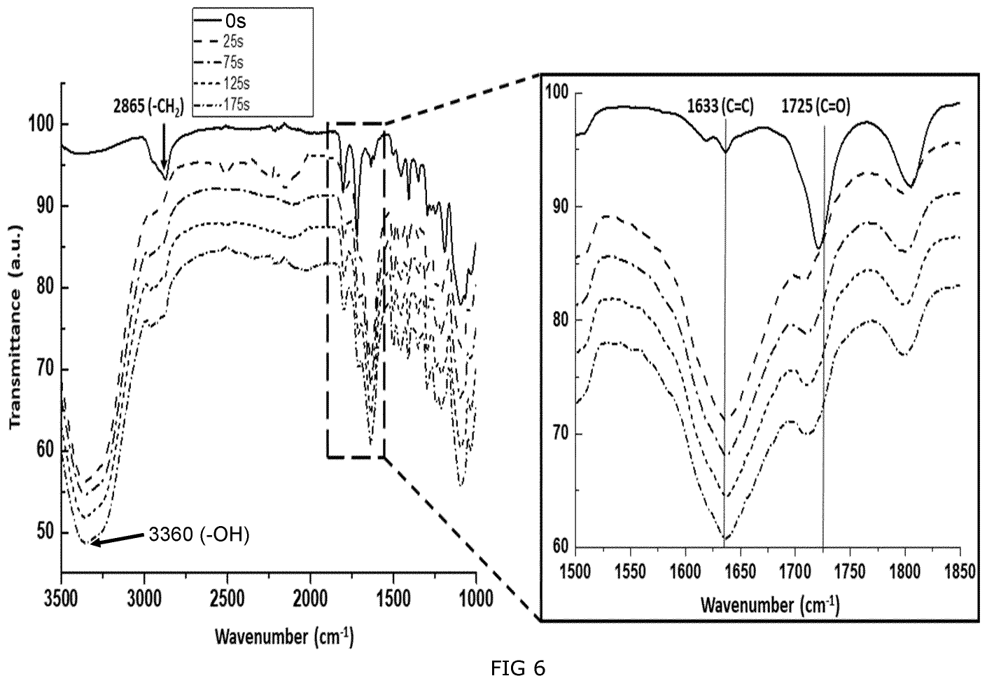

Polymerisation of PEGDA monomer and subsequent swelling was assessed with TR-FTIR spectroscopy. The unreacted device showed a peak at 2856 cm⁻¹ corresponding to unreacted (CH₂) terminal group of the PEGDA monomer (FIG. 6). The peak disappears as the reaction proceeds upon addition of water. Polymer chain extension was confirmed with a shifting carbonyl band (1730 cm⁻¹) of the acrylate group and characteristic hydrogel swelling (OH peak) at 3360 cm⁻¹. This peak was absent prior to the reaction.

### Example 4: In Vivo Rat Study

The devices were loaded into a Torpac® size 9 gelatin capsule which was coated in a 12% (w/v) Eudragit L100 solution in IPA with dibutyl sebacate as plasticizer in a 5% w/w ratio relative to Eudragit. Each size 9 capsule contained ca. 600 devices. Male Sprague-Dawley rats (Janvier Labs, Le Genest-Saint-Isle, France) were housed in groups of six per cage and allowed to acclimatize for at least one week with a reversed 12/12 h day/night cycle. Fasting of the rats was initiated 12-16 h prior to the studies and the study was conducted under full anaesthesia. The abdominal cavity was opened after which an enteric-coated capsule with a magnet inside was administered to the stomach using a gavage dosing tube. The capsule was dragged to the duodenum using an external magnet. The rats (n=2) were kept anesthetized for 3 h, after which they were euthanized by an intracardiac injection of pentobarbital (100 mg/kg). The experiments were carried out in concordance with the Danish law on animal experiments as approved by the Danish Animal Experiments Inspectorate in accordance with the EU directive. Since the aim of the study was to verify devices activation in vivo, and subsequent entrapment of gut microbiota in the hydrogel matrix, excised animal tissue and mucus layer served as a control. After euthanasia, the stomachs and small intestines were removed from the rats that had been administered devices in order to localize the position and orientation of the devices (stored at −20° C.).

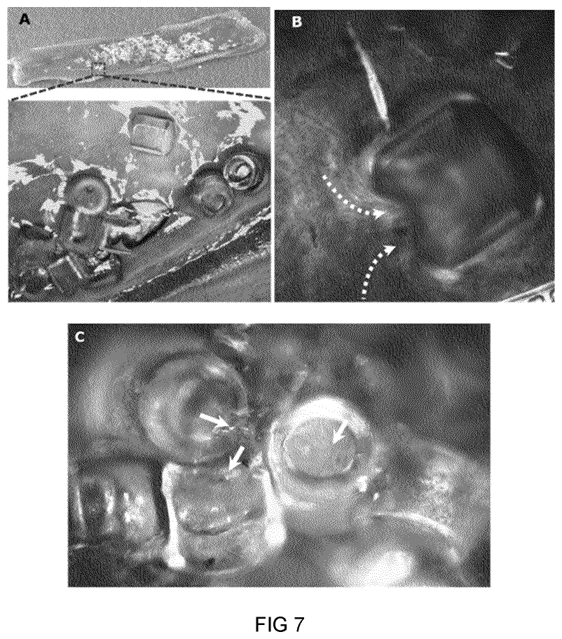

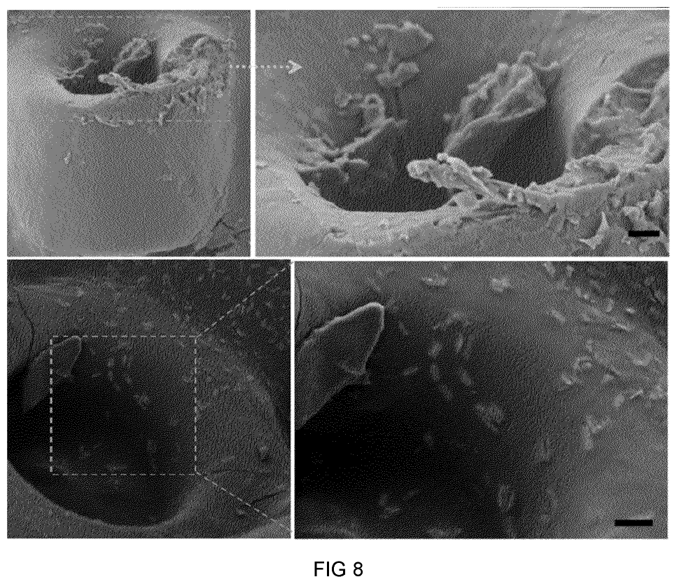

The devices successfully deployed inside the rats' GI tracts, as confirmed by visual inspection (FIG. 7) and by SEM imaging of the excised tissue. The SEM images showed the formed hydrogel (FIG. 8), as well as bacteria adsorbed onto the device.

### Example 5: Proteolytic Enzyme Activity in Devices (Post-In Vivo Administration)

The proteolytic enzyme activity was assessed using a trypsin-based assay wherein trypsin from trapped cells cleaves a succinylated casein substrate to generate peptide fragments with free amino-terminal groups. These peptides can react with 2,4,6-trinitrobenzenesulfonic acid (TNBSA) forming a coloured TBN-peptide product. Pierce™ Protease Assay Kit was obtained from Thermo Scientific, Denmark. 50 mM, pH 8.5 assay buffer was prepared by dissolving the contents of BupH Borate Pack in 500 mL distilled water. Lyophilized Succinylated Casein was dissolved in assay buffer to get 2 mg/mL Succinylated Casein solution. Lyophilized TPCK Trypsin was then dissolved in assay buffer to prepare a 50 mg/mL stock solution. Sample was stored at −80° C. TN BSA stock was diluted in assay buffer to make TNBSA working solution. Trypsin stock solution was diluted to 0.5 mg/mL in Assay buffer. The stock solution was used to prepare serial dilutions of 0.1, 0.2, 0.3, 0.4, 0.5 mg/mL. 100 μL Assay Buffer was added into the first well in 96-well plate (TS Varioskan Multiplate reader), serving as a blank control. 100 μL Succinylated Casein Solution was pipetted into the next five wells, followed by the addition of 50 μL serial dilutions in proper order. The plate was incubated for 20 minutes at 37° C. in the drying oven. Then 50 μL of TNBSA Working Solution was added into each well and the plate was incubated for another 20 minutes at room temperature. The change in Trypsin activity was detected by measuring the absorbance at 450 nm (ΔA₄₅₀) with constant shaking (pulsed) at 600 rpm. A standard curve was plotted of the absorbance at 450 nm as a function of concentration, providing a linear correlation with R²=0.99368.

The devices were recovered from the ileum tissue and washed with acetate buffer (pH 4) to remove adhered mucus, followed by PBS, and distilled water, each washing was done three times (i.e. a total of nine washes). The cleaned devices were ready for the trypsin activity testing. The study was performed using the same method as is described for trypsin activity standard curve, except instead of the serial dilutions, 50 μL Assay Buffer containing an increasing number of devices (1, 3, 5, 7, 9) was used. The well with no devices was set as blank control. A duplicated set of wells was prepared and the absorbance at 450 nm wavelength (ΔA₄₅₀) was measured with constant shaking (pulsed) at 600 rpm.

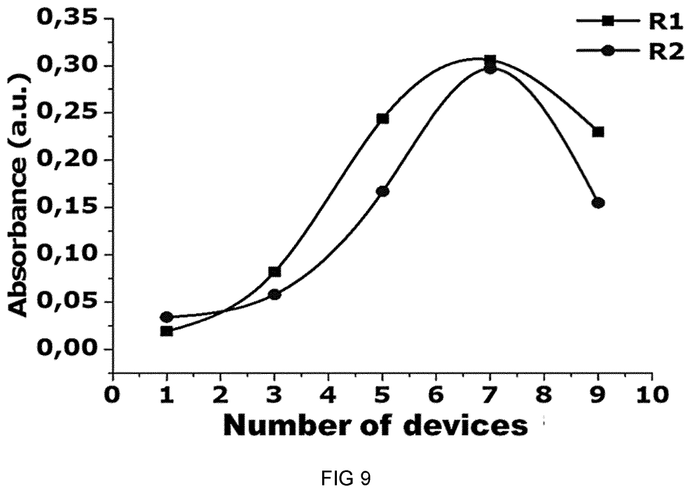

Trypsin activity gradually increased with increasing number of device (FIG. 9). Nonetheless, a decrease in enzymatic activity upon reaching a certain threshold (7-9 devices) was observed, which can be attributed to dimerization of proteolytic enzymes as well as steric hindrances owing to the presence of high amount of enzymes in a small reaction volume. It is considered that the devices of the present disclosure is able to collect other biomarkers as well.

### Example 6: Sample Preparation for qPCR Sequencing

The devices were taken from the ileum tissue and washed thrice, each with acetate buffer (pH 4), PBS, 70% EtOH, 2 minutes each with vigorous vortex—i.e. a total of nine washes for each device. Control sample consisted of scrapped mucosa with devices from the same ileum sample but were not washed or pre-treated in any manner. The devices were cracked open via a ball milling process and the sample was extracted using the Invitrogen Purelink biome kit using the standard protocol. The sequencing library was prepared using the standard Illumina Nextera protocol. The regions that were amplified in the primary PCR were the V3-V4 16S rRNA gene regions. The raw sequencing reads were run through the standard BION pipeline. Reads not belonging to bacteria were filtrated from the dataset.

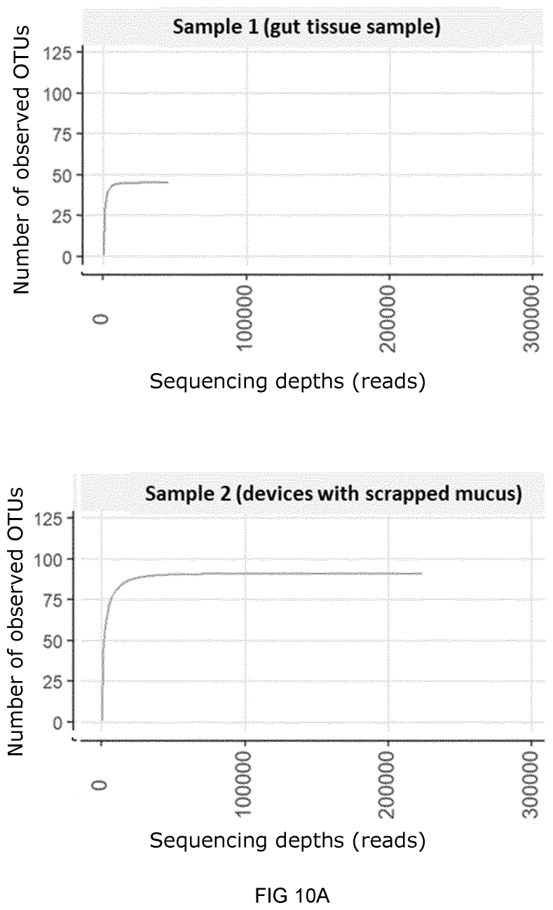

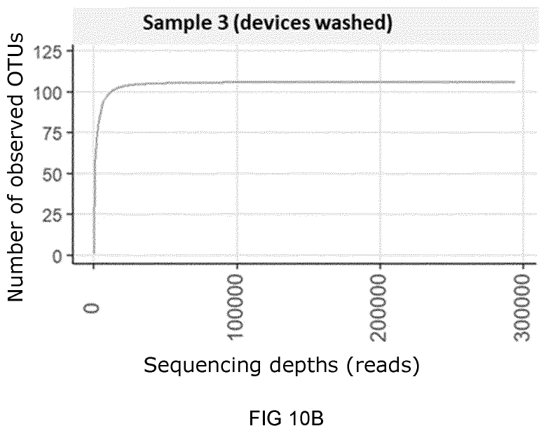

The thorough washing ensured that only the microbial DNA that was enmeshed within the hydrogel matrix was amplified for genome sequencing. The rarefaction curves at genus level indicated sufficient sequencing depths as highlighted in FIG. 10. Table 1 describes different alpha diversity indexes at genus level for each sample. The lowest number of reads were 294135 and the samples were rarefied at 294135 reads to compare the alpha diversity of the samples. Similar to the rarefaction curve (FIG. 10), lowest number of OTUs was observed in the ‘gut tissue sample’ (sample 1), followed by ‘device with scrapped mucus’ (Sample 2), and highest in ‘washed device’ (sample 3). This was also represented in the Shannon diversity index indicating that sample 1 has lower species richness as compared to sample 2 and sample 3, that few genera dominates in sample 1.

TABLE 1
Alpha diversity indexes at genus level for
samples from scrapped mucus and device.

| | Total | Raw | Observed | |
|---|---|---|---|---|
| Sample | reads | Reads | OTUs | Shannon |
| 1. Gut tissue sample | 102881 | 45054 | 45 | 1.982535 |
| 2. Device (with | 257868 | 223787 | 91 | 2.391751 |
| scrapped mucus) | | | | |
| 3. Washed device | 308493 | 294135 | 106 | 2.792572 |

The results presents different identified bacterial groups as shown in Table 2. Sample 1 was dominated by five genera: Listeria, Bifidobacterium, Lactobacillus and Romboutsia. All these microbes are also the original residents of rat's GI tract (small intestine). Lactobacillus and Bifidobacterium spp. are responsible for short-chain fatty acid (SCFA) production in murine model. Murine models are carrier of Listeria spp. in their gut and results in foodborne illness in human beings. Romboutsia sp. is a natural and abundant inhabitant of the rat small intestine and is a non-motile obligate anaerobe, which generally resides under the thick mucus layer. Further, all these bacteria were also detected in sample 2 and sample 3 (except Bifidobacterium). Absence of Bifidobacterium in Sample 3 can be attributed to removal during the washing step. Variation in microbial population was anticipated and observed due to three main reasons: i) varying zone of activation for each individual device; ii) Heterogeneity of GI microbiota within the same GI region and varying mucus layer thickness; and iii) total number of devices retrieved post-activation for genomic analysis. These findings show the ability of the technology to sample gut microbiota via an orally ingestible device.

TABLE 2
Abundance of gut microbiota at genera level.

| | | Sample 2 | |
|---|---|---|---|
| | | Device (with | Sample 3 |
| | Sample 1: | scrapped mucus | Device |
| | Rat gut tissue | layer) | (washed) |
| Listeria | 32.2 | 4.1 | 0.5 |
| Bifidobacterium | 25.9 | 1.7 | 0 |
| Lactobacillus | 10.2 | 12.1 | 5 |
| Bacillus | 9.8 | 0.3 | 0.4 |
| Romboutsia | 9.2 | 27.3 | 0.2 |
| Staphylocaccous | 4.3 | 0.3 | 1.2 |
| Corynebacterium | 1.7 | 1.9 | 2.6 |
| Propionibacterium | 1.1 | 0.4 | 1.2 |
| Lactococcus | 0.9 | 0.3 | 0.7 |
| Escherichia | 0.7 | 0 | 0 |
| Pseudomonas | 0.5 | 1.7 | 1.9 |
| Faecalibaculum | 0.4 | 0.3 | 0 |
| Enterococcus | 0.3 | 1.7 | 0 |
| Leuconostoc | 0.2 | 10.4 | 17.1 |
| Micrococcus | 0.2 | 0.2 | 0.1 |

### Example 7: In Situ Polymerisation of Hydrogel Pre-Polymer Improves Bacterial Absorption

Materials and Methods

E. coli DH5α was cultured overnight in LB media, centrifuged, and washed thrice with PBS. The pellet was suspended in PBS with OD 600=0.6. A hydrogel forming reaction comprising of sodium polyacrylate (200 mg), ascorbic acid, and iron trichloride (25 mg, dry wt. ratio 5:1), was packed in small centrifuge tubes (eight in number). Four tubes were allocated as ‘control’, where the mixture would be polymerized before bacterial introduction, and Four tubes were marked as ‘reaction’ for bacterial enmeshing via hydrogel formation. Bacterial suspension (1.5 mL) was introduced in the ‘reaction’ set for a period of 2 minutes to initiate gel formation, and washed twice with PBS. The ‘control’ set was pre-activated with PBS for a 2 minutes, and upon gel formation, bacterial suspension (1.5 mL) was introduced for 2 minutes, and washed twice with PBS.

The resulting gel was introduced to the DNA extraction process. 3 mL of Lysis buffer (PureLink®) was used to disrupt the bacterial cells in both reaction sets with gentle mechanical crushing with a spatula for 5 minutes. This was followed with addition of Precipitation Buffer and all the tubes were centrifuged at 12,000×g for 10 minutes. The supernatant was loaded in QIAmp UCP Mini Spin column for binding followed by washing twice with the Wash Buffer. The wash through was discarded and gently centrifuged to further discard excess buffer. Elution buffer was added to each tubes to extract the DNA followed by isopropanol precipitation. All microtubes were centrifuged at 12,000×g for 5 minutes at 4° C. and the supernatant was discarded. The purified genomic DNA pellet was suspended in TE Buffer (50 μL). Finally, extracted DNA concentration was measured with a Nanodrop® instrument (260 nm) (Table 3).

TABLE 3
Average DNA entrapped in hydrogel

| S. No. | Sample name | DNA Average (ng/μL) |
|---|---|---|
| 1. | Reaction set (self- polymerizing) | 15.95 |
| 2. | Control (pre-polymerized) | 4.7 |

Results

The In situ hydrogel forming system demonstrated over three times higher DNA yield indicating higher bacterial entrapment.

Conclusion

The results above indicates that the in situ hydrogel forming system entraps significantly more bacteria than an already polymerised hydrogel.

### Example 8: Trapping of Fluorescent Microparticles in Device Featuring Physical Separator

A capsule-half design was constructed as shown in FIG. 11. The design comprised pre-polymer loading chambers (1, 3) and a physical separator 2 having an opening. FIGS. 11B and C show hydrogel formation of sodium polyacrylate and iron chloride/ascorbic acid in chambers 1 (FIG. 11B) and 3 (FIG. 11C), respectively. Light spots correspond to green fluorescence from fluorescent microparticles incorporated into the PBS buffer used for activation of hydrogel formation. The fluorescence demonstrates the particle trapping. An air bubble present in chamber 3 rose towards opening in separator 2 upon polymerisation, physically separating chambers 1 and 3, thereby minimizing cross contamination.

### Example 9: Further Device Designs

Further device designs are envisioned in FIG. 12. Dashed round shapes indicate openings. The half-capsule devices shown on the figure can seamlessly fit into a standard gelatin capsule-half. The standard gelatin capsule-half can be enteric-coated to disintegrate at specific pH of the GI tract, thereby, releasing the half-capsule device. FIG. 12A shows a device featuring chambers 4 and 6 separated by separator 5. A funnel-like feature (dashed, straight lines) ensures complete loading of pre-polymermixture inside the inner chamber 6. FIG. 12B shows a device featuring dissolvable blocks 8 preventing spring-based-bead closing mechanism 9 from closing against stoppers 7. Upon dissolution of blocks 8, spring-based-bead mechanism 9 closes against stoppers 7. The pre-polymer mixture is loaded in the device above and/or below blocks 8. Upon entry of luminal fluids into device, pre-polymer mixture swells and/or polymerises to enmesh biomarkers and/or microbiome, while stopper 8 gets dissolved, to release the spring of spring-based-bead closing mechanism 9 and blocking the channel in stoppers 7. Polymerized and swollen hydrogel may create a positive pressure inside the device and may seal the opening to minimize cross-contamination during the transit of the GI tract. FIG. 12C shows a device with spring-based closing mechanism featuring lid 13 connected to stoppers 10 by extended springs 11, held extended by dissolvable blocks 12. Upon dissolution of blocks 12, lid 13 closes against stoppers 10. The tension and/or length of springs 11 can be varied to control the volume of the fluids entering in the inner chamber for polymerization. Hydrogel pre-polymer is present in the device under lid 13. Polymerized hydrogel creates a positive pressure inside the device and seals the entry points to minimize cross-contamination during the transit. FIG. 12D shows a device having one end-opening 14 and a solid base 17, a pre-polymerization mixture loading area 16, and a lid 15. Upon polymerisation and expansion of hydrogel pre-polymer, lid 15 closes against opening 14.

1. An oral delivery capsule comprising:
a. a non-biodegradable, biocompatible open container with an extraction component and at least one opening;
b. a dry composition of a radical initiator and a hydrogel pre-polymer; and
c. an enteric coating;
wherein the dry composition is inside the container and the enteric coating covers the opening of the container.

2. The oral delivery capsule according to claim 1, wherein the hydrogel pre-polymer comprises PEGDA.

3. The oral delivery capsule according to any one of the preceding claims, wherein the hydrogel pre-polymer is PEGDA.

4. The oral delivery capsule according to any one of the preceding claims, wherein the PEGDA has a molecular weight of 400 to 750 g/mol, such as about 575 g/mol.

5. The oral delivery capsule according to any one of the preceding claims, wherein the radical initiator comprises iron trichloride.

6. The oral delivery capsule according to any one of the preceding claims, wherein the radical initiator comprises ascorbic acid.

7. The oral delivery capsule according to any one of the preceding claims, wherein the radical initiator is a mixture comprising iron trichloride and ascorbic acid.

8. The oral delivery capsule according to any one of the preceding claims, wherein the radical initiator and the pre-polymer are heterogeneously comprised within the container.

9. The oral delivery capsule according to any one of the preceding claims comprising a non-biodegradable, biocompatible open container, a first body of radical initiator, and a second body of pre-polymer, wherein the first body of radical initiator is encapsulated fully by the combination of non-biodegradable, biocompatible open container and the second body of pre-polymer.

10. The oral delivery capsule according to any one of the preceding claims, wherein the non-biodegradable, biocompatible open container is made of a non-biodegradable and biocompatible polymer.

11. The oral delivery capsule according to any one of the preceding claims, wherein the non-biodegradable, biocompatible polymer is SU-8.

12. The oral delivery capsule according to any one of the preceding claims, wherein the non-biodegradable, biocompatible polymer is PLA.

13. The oral delivery capsule according to any one of the preceding claims, wherein the non-biodegradable, biocompatible open container is cylindrical.

14. The oral delivery capsule according to any one of the preceding claims, wherein the non-biodegradable, biocompatible open container comprise one or two openings.

15. The oral delivery capsule according to any one of the preceding claims, wherein the non-biodegradable, biocompatible open container is cylindrical and comprise two openings, such as a cylinder having two open end faces.

16. The oral delivery capsule according to any one of the preceding claims, wherein the non-biodegradable, biocompatible open container is cylindrical and comprise one opening, such as a cylinder having one closed end face and one open end face.

17. The oral delivery capsule according to any one of the preceding claims, wherein the container further comprises a lid element.

18. The oral delivery capsule according to any one of the preceding claims, wherein the opening and/or the closing of the lid element is controlled by a lid mechanism.

19. The oral delivery capsule according to any one of the preceding claims, wherein said lid mechanism comprises or more springs.

20. The oral delivery capsule according to any one of the preceding claims, wherein said lid mechanism is activated by dissolution of a dissolvable block.

21. The oral delivery capsule according to any one of the preceding claims, wherein said dissolvable block comprises alum.

22. The oral delivery capsule according to any one of the preceding claims wherein the oral delivery capsule comprises a container having one end opening capped with a capsule cap.

23. The oral delivery capsule according to any one of the preceding claims having a length between 5 mm and 30 mm and a diameter between 1 mm and 13 mm.

24. The oral delivery capsule according to any one of the preceding claims, wherein the container has a length between 5 and 30 mm, a height between 1 and 13 mm, and a width between 1 and 13 mm.

25. The oral delivery capsule according to any one of the preceding claims, wherein the container is cylindrical and has a length between 5 and 30 mm and a diameter between 1 and 13 mm.

26. The oral delivery capsule according to any one of the preceding claims having a length between 7 and 25 mm and a diameter between 1 and 3 mm.

27. The oral delivery capsule according to any one of the preceding claims having a length between 20 and 30 mm and a diameter between 9 and 13 mm.

28. The oral delivery capsule according to any one of the preceding claims comprising exactly one container.

29. The oral delivery capsule according to any one of the preceding claims, wherein the capsule further comprises a contrast agent.

30. The oral delivery capsule according to any one of the preceding claims, wherein the container further comprises a contrast agent.

31. The oral delivery capsule according to any one the preceding claims, wherein the contrast agent is a barium based or gadolinium based contrast agent.

32. The oral delivery capsule according to any one of the preceding claims, wherein the contrast agent is barium sulfate or gadopentetic acid.

33. The oral delivery capsule according to any one of the preceding claims, wherein the extraction component is a magnetic material.

34. The oral delivery capsule according to any one of the preceding claims, wherein the extraction component is a coloured material.

35. The oral delivery capsule according to any one of the preceding claims, wherein the extraction component is encapsulated in the material of the non-biodegradable, biocompatible open container.

36. The oral delivery capsule according to any one of the preceding claims, wherein the at least one opening of the container is covered in an enteric coating.

37. The oral delivery capsule according to any one of the preceding claims, wherein the open container having on the inside a dry composition of a radical initiator and a hydrogel pre-polymer is covered in an enteric coating.

38. The oral delivery capsule according to any one of the preceding claims, wherein the capsule is covered in an enteric coating.

39. A method for sampling microbiome in vivo, said method comprising
a. polymerising a hydrogel pre-polymer in the gut, thereby absorbing microbiome into the polymerised hydrogel, and
b. collecting the polymerised hydrogel.

40. A device comprising:
a. a non-biodegradable, biocompatible open container with an extraction component and at least one opening;
b. a dry composition of a radical initiator and a hydrogel pre-polymer; and
c. an enteric coating.

41. The device according to claim 40, further comprising a lid opening and closing mechanism.

42. The device according to claim 41, further comprising an actuator component controlling the lid opening and closing mechanism.

43. A non-biodegradable, biocompatible open container with at least one opening, having a swollen hydrogel polymer comprising intestinal microbiome.

44. The non-biodegradable, biocompatible open container with at least one opening of claim 43, further having an extraction component.

45. Use of an oral delivery capsule comprising a non-biodegradable, biocompatible open container with at least one opening, having on the inside a dry composition of a radical initiator and a pre-polymer, wherein the at least one opening of the open container and/or the capsule is covered in an enteric coating, for collection of the intestinal microbiome in a subject.

46. A method for collecting microbiome of the jejunum and/or ileum, said method comprising the steps of:
a. providing an oral delivery capsule comprising a open container having on the inside a hydrogel pre-polymer and a radical initiator, wherein said container having on the inside a hydrogel pre-polymer and a radical initiator and/or the capsule is covered in an enteric coating;
b. ingesting the oral delivery capsule;
c. dissolving the enteric coating in the jejunum and/or ileum, effecting polymerisation of the hydrogel pre-polymer to produce a biocompatible hydrogel,
d. absorbing the microbiome present in the vicinity of the container into the hydrogel,
e. excreting the container comprising the hydrogel and absorbed microbiome, and
f. isolating the container comprising the hydrogel and absorbed microbiome from the excretion.
